(19) United States
(12) Patent Application Publication
(10) Pub. No.: US 2009/0142840 A1
(43) Pub. Date: Jun. 4, 2009

(54) DNA encoding anti-apoptotic protein and recombinant 30K protein

(75) Inventors: Tai Hyun Park, Seoul (KR); Eun Jeong Kim, Seoul (KR); Hye Jung Park, Suwon (KR)

Correspondence Address: THE NATH LAW GROUP, 112 South West Street, Alexandria, VA, 22314, US

(73) Assignee: HANSON BIOTECH CO., LTD., Daejeon (KR)

(21) Appl. No.: 12/222,338

(22) Filed: Aug. 7, 2008

Related U.S. Application Data
(63) Continuation of application No. 10/926,406, filed on Aug. 26, 2004, now abandoned.

(51) Int. Cl. C12N 15/09; C07K 14/00

(52) U.S. Cl. 435/455; 530/350

(57) ABSTRACT

The present invention relates to DNAs encoding anti-apoptotic 30K proteins. More particularly, the present invention is directed to 30K protein genes and a recombinant proteins prepared by using novel anti-apoptotic gene obtained from silkworm. The present invention also provides anti-apoptotic health care food, pharmaceutical preparation, additive for cell culture medium, and food supplement.

### FIELD OF INVENTION

The present invention relates to DNAs encoding anti-apoptotic protein and a recombinant 30K protein. More particularly, the present invention is directed to novel anti-apoptotic DNAs obtained from silkworm and a recombinant 30K protein.

### DESCRIPTION OF THE RELATED ART

Apoptosis is a normal physiologic process that leads to individual cell death. This process of programmed cell death is involved in a variety of normal and pathogenic biological events and can be induced by a number of unrelated stimuli.

Changes in the biological regulation of apoptosis also occur during aging and are responsible for many of the conditions and diseases related to aging. Recent studies of apoptosis have implied that a common metabolic pathway leading to cell death may be initiated by a wide variety of signals, including hormones, serum growth factor deprivation, chemotherapeutic agents, ionizing radiation, and infection by human immunodeficiency virus (HIV) (Wyllie (1980) Nature 284:555-556; Kanter et al. (1984) Biochem. Biophys. Res. Commun. 118:392-399)

Apoptosis occurs sporadically in all tissues throughout life and is a normal everyday occurrence; however, disproportionate apoptosis, either excessive or deficient may cause serious diseases.

Many researchers have found that several extant diseases associated with apoptosis, particularly relates to cancer and autoimmune disease which were caused by deficiency in apoptosis, and dementia and Alsheimer's disease which were caused by surplus apoptosis, and so called a degenerative disease and AIDS.

Subsquent researches have been developed for clinical trial to treat above described by means of these anti-apoptotic gene and proteins, or to use factors intervening signal transduction, that induces apoptosis. These researches were concentrated on regulation of apoptosis, induction of apoptosis, and biological mechanism.

Recently, it has been known to the public that several genes such as Bcl-2 family inhibit the apoptosis effectively.

In actual, the study has been preceded to inhibit the necrosis and apoptosis of PC12 cell induced by amyloid peptide related to the demetia of the aged (Neurosci of Apoptosis Protein:IAP). Also, to treat cancer, antisense technology of bcl-2, hsp27 has been tried. Especially, treatment by anti-bcl-2 is known to be effective in lymph tumor (J. Natl, Cancer Inst. 89,998(1997), Lancet 349,1137(1997)).

By several result of study until now, as many regulative factors of apoptosis have found, technology using regulator or genome information of it has been developed from two points of view.

One point is related to the study for treatment of the disease induced by cell death. Second point is related to the study of cell culture improving cell-production by inhibiting apoptosis. These studies have been done by using bcl-2 family proteins and genes.

The present inventors have conducted intensive researches in regard to anti-apoptotic factor existing in silkworm hemolymph.

As a result, the present inventors have discovered a novel method of fractionation, seperation, purificaton of proteins obtained by silkworm hemolymph, which are capable to substitute for conventional anti-apoptotic proteins encoded by genes such as bcl-2, and that productivity of the recombinant protein and the viability of host cells increase in insect/baculovirus system with adding silkworm hemolymph to the culture medium (Biotechnol. Prog., 15, 1028 (1999)).

Also, the present inventors have found that silkworm hemolymph has inhibitory factor of apoptosis, and found that silkworm hemolymph inhibits not only virus-induced apoptosis but also apoptosis induced by various chemicals. Moreover, silkworm hemolymph also inhibits human cell apoptosis.

The present inventor has also found these active factor is a kind of protein, which is seperated from silkworm hemolymph (Korean Patent Application No. 10-2001-0010717, Biochem. Biophys. Res. Commun., 285, 224 (2001)). The corresponding genes are obtained by Polymerase Chain Reaction (PCR) with the primers designed using information of the above purificated protein, and the gene sequence is analyzed.

The result of the gene sequence analysis indicated that the gene is so called “30K protein” of which function had not yet been known. The 30k protein group consists of five proteins and have the sequence Id. No.1 to 5, respectively.

Also it had been known to the public that 30K proteins have common characteristics in amino acid composition and immunological activity as well as molecular weight and they are a group of structurally related proteins with a molecular mass of approximately 30,000 Da.

The genes encoding the 30K proteins were remarkably different from the anti-apoptotic proteins such as bcl-2 family, which had been known to the public.

The object of the present invention is to provide anti-apoptotic protein originating from silkworm hemolymph. To accomplish the object efficiently, recombinant DNA technology is used to produce useful recombinant proteins in the present invention.

The pET-22b(+) carrying the 30K protein gene is introduced into E. coli BL(DE3). The 30K protein obtained from recombinant cell is proven to have an effect on inhibiton of apoptosis.

According to the present invention, the anti-apoptotic protein enables us to produce pharmaceuticals and health care food. For example, the anti-apoptotic recombinant protein is effective to dementia and Alsheimer's disease, which may be caused by surplus apoptosis, and so called a degenerative disease and AIDS as treatment. Also, the anti-apoptotic recombinant protein of the present invention is applied to food additives and cell-culture medium additives for improving productivity of incubating cells. On the basis of the above discoveries, the inventors could have accomplished the present invention.

Korean Patent Application No. 10-2001-0010717, No. 10-2002-0059686 are referenced in this application in order to more fully describe the state of the art to which this invention pertains. The disclosure of each of these publications and patent documents is incorporated by reference herein.

### SUMMARY OF THE INVENTION

Therefore, the primary purpose of the present invention is to provide DNA SEQ. ID. No. 1 encoding the 30Kc6, DNA SEQ. ID. No. 2 encoding the 30Kc12, DNA SEQ. ID. No. 3 encoding the 30Kc19, DNA SEQ. ID. No. 4 encoding the 30Kc21 and DNA SEQ. ID. No. 5 encoding the 30Kc23.

It is an another object of the present invention to provide anti-apoptotic recombinant proteins comprising an amino acid sequence set forth as SEQ. ID. No.6, ID. No.7, ID. No.8, ID. No.9 and ID. No.10.

It is a still another object of the present invention to provide an anti-apoptotic pharmaceutical preparation comprising a therapeutically effective amount of the recombinant anti-apoptotic protein in a pharmaceutically acceptible carrier.

It is a yet another object of the present invention to provide an anti-apoptotic health food, additive for culture medium and food supplement comprising the recombinant protein.

It is a further object of the present invention to provide a recombinant expression vector comprising the DNA of SEQ. ID. No.1 encoding anti-apoptotic 30Kc6 protein, ID. the DNA of SEQ. No.2 encoding anti-apoptotic 30Kc12 protein, the DNA of SEQ. ID. No.3 encoding anti-apoptotic 30Kc19 protein, ID. the DNA of SEQ. No.4 encoding anti-apoptotic 30Kc21 protein and ID. the DNA of SEQ. No.5 encoding anti-apoptotic 30Kc23 protein.

### BRIEF DESCRIPTION OF THE DRAWINGS

The above objects and other advantages of the present invention will become more apparent by describing in detail a preferred embodiment thereof with reference to the attached drawings, in which:

FIG. 1A to FIG. 1C are the photographs of electrophoresis, which indicate that the recombinant 30K protein expressed in E. coli BL21 (DE3) (A), BL21 (DE3) (B) and purified recombinant 30K protein (C).

FIG. 2A to FIG. 2B are the graphs which show the viability of host cell measured 7 days after virus infection in the media supplemented with the recombinant 30K protein described in the above FIG. 1(c).

FIG. 3A to FIG. 3D are the FACS analytic chromatogram, which show the effect of 30K protein on actinomycin D-induced insect cell apoptosis. Except for (a), the cells were treated with 200 ng/ml actinomycin D for 13 h and apoptosis was analyzed by flow cytometry. (A) Sf9 cells not treated with actinomycin D, (B) Sf9 cells cultured in the medium containing 10% FBS, (C) Sf9 cells cultured in the medium containing 5% FBS and 5% hemolymph, and (D) Sf9 cells cultured in the medium containing 5% FBS and 0.2 mg/ml recombinant 30K protein.

FIG. 4A(a) to FIG. 4A(C) are the photographs which show the effect of 30K protein on staurosporine-induced human cell apoptosis. HeLa cells were treated with 600 nM staurosporine for 12 h and apoptosis was analyzed by fluorescence microscopy using Hoechst 33258 fluorescent dye. (A): (a) Cells cultured in the medium containing 5% FBS and 5% hemolymph. (c) Cells cultured in the medium containing 5% FBS and 0.2 mg/ml recombinant 30K protein.

FIG. 4B is the graph, which indicates percentage of apoptotic cells represented in (B) was determined by counting the number of apoptotic cells, which were detected by the method used in (A).

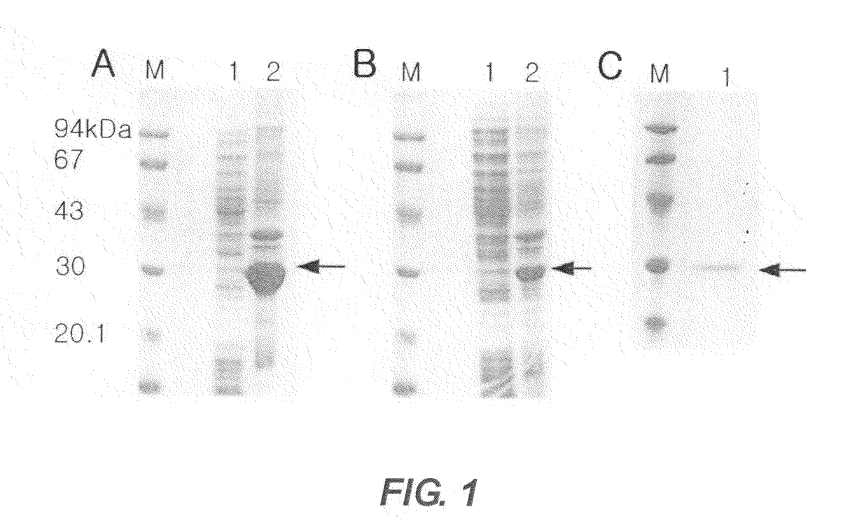

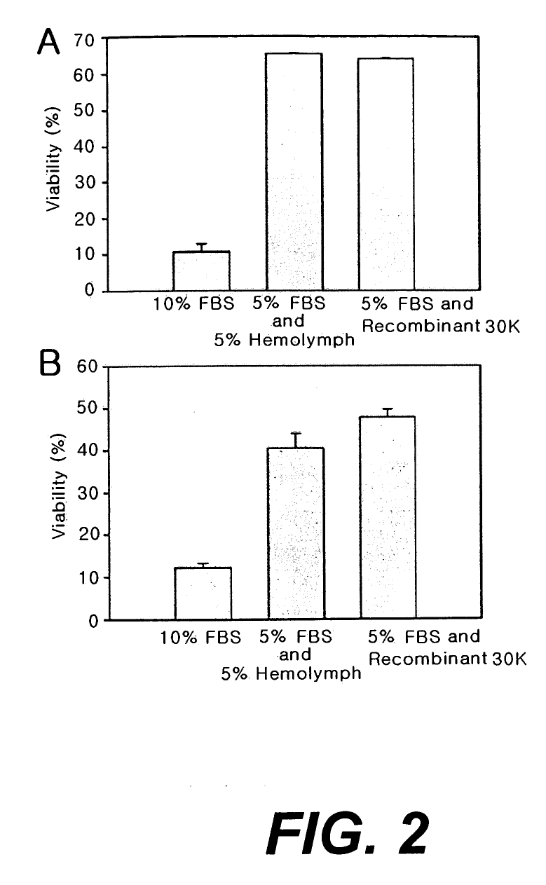

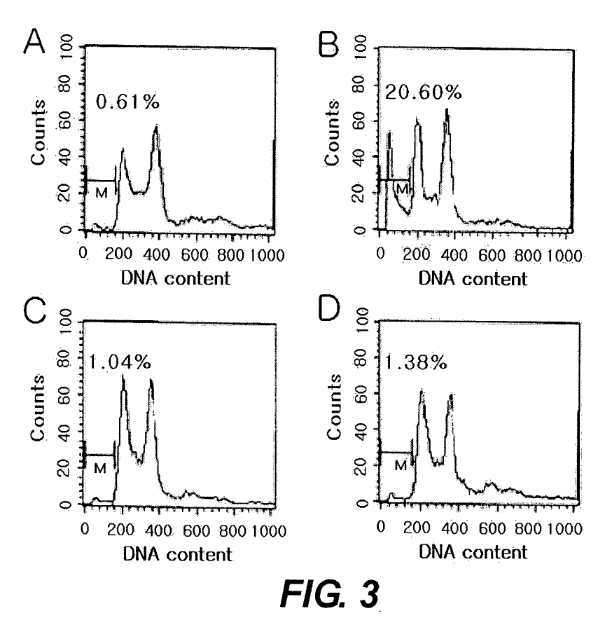

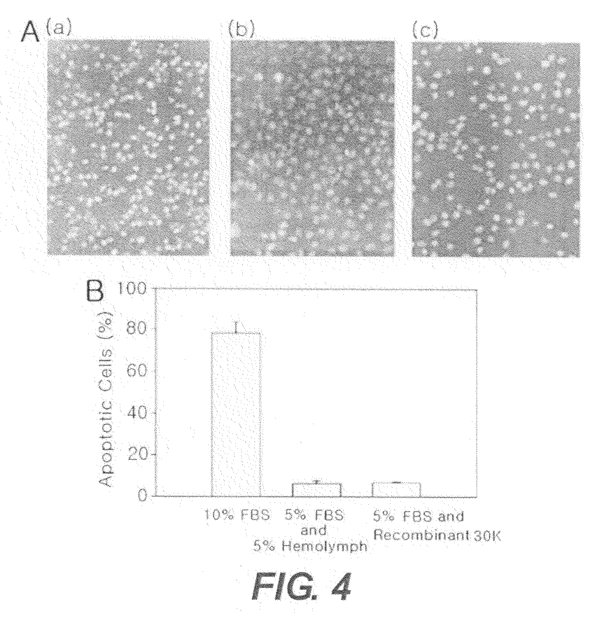

### DETAILED DESCRIPTION OF THE INVENTION

Cell death is categorized as either apoptotic or necrotic. Apoptosis is a physiological cell death, which is morphologically distinguishable from necrosis.

Necrotic cells are characterized by an overall increase in size, mild clumping of chromatin and cell lysis.

However, apoptosis is different from necrosis where healthy cells are destroyed by external processes, such as inflammation. Apoptosis is a kind of voluntary, programmed death of cells that is under genetic control. The cell's own genes play an active role in its demise and is accompanied by the condensation of nuclei and cytoplasm, the loss of microvilli, convolution of the plasma membrane, and nuclear and cell segmentation.

Therefore, above objection of the present invention is achieved by providing an anti-apoptotic recombinant anti-apoptotic protein and DNAs encoding anti-apoptotic 30Kc6, 30Kc12, 30K19, 30K21, and 30K23 protein. It enables apoptosis to be inhibited effectively in animal cells and human cells.

In one embodiment of the present invention, there is provided anti-apoptotic protein synthesized by genetic recombination technology using gene of protein separated from silkworm homolymph.

DNAs of SEQ. ID. No.1 to 5 encoding anti-apoptotic 30Kc6, 30Kc12, 30K19, 30K21, and 30K23 protein, are obtained from silkworm, repsectively.

A silkworm hemolymph has been used effectively in biological researches. The production of recombinant protein in an insect cell baculovirus system was increased by supplementing the medium with silkworm hemolymph. Silkworm hemolymph increases baculovirus-infected insect cell longevity.

Moreover, it has been shown that silkworm hemolymph inhibits apoptosis in insect, mammalian, and human cell systems. These results indicate that silkworm hemolymph contains a component that inhibits apoptosis.

More recently, this anti-apoptotic fraction was isolated from silkworm hemolymph and characterized by the present inventors.

The fraction of silkworm hemolymph with the highest activity was found to contain 30K proteins, which are a specific type of plasma protein called “storage proteins”. These proteins constitute a group of structurally related proteins of approximate molecular weight 30,000 Da. The 30K protein group consists of five proteins, which have common characteristics in amino acid composition and immunological activity as well as molecular weight.

The 30K protein encoded by the 30Kc6 gene of the present invention was expressed in Escherichia coli and purified. E.coli BL21 (DE3) was used as the host for gene expression in the present invention.

Total RNA was isolated from silkworm tissue using RNA isolation kit, and total cDNA pool was obtained by RT-PCR using an oligo-dT primer. The 30K protein cDNA was amplified from the cDNA pool by PCR using specific primers. Then the amplified PCR products were cloned into E. coli expression vector, pET-22b(+). During this step a signal sequence contained in 30Kc6 was removed, and the vector was designed to express the 30K protein with a 6^{x} His tag at its C-terminal. E.coli BL21 (DE3) was used as the host for gene expression.

Hereinafter, the present invention will be described in greater detail with reference to the following examples. The examples are given for illustration of the invention and not intended to be limiting the present invention.

### EXAMPLE 1

Plasmid Containing 30K Protein cDNA Construction

The 30Kc6(GenBank Accession No.: X07552) protein cDNA was amplified by PCR with a temperature profile of 95° C. for 1 min, 56° C. for 1 min, and 72° C. for 1.5 min.

The forward and reverse primers were 50-AGA CAT ATG ACA CTT GCA CCA AGA ACT-30 and 50-CAA CTC GAG GTA GGG GAC GAT GTA CCA-30, respectively, which contain the NdeI and XhoI sites, respectively. The forward primer contains ATG for methionine, which is necessary for the initiation of translation in E. coli.

The amplified PCR products were cloned into a NdeI-XhoI site in E.coli expression vector, pET-22b(+). During this step, we removed a signal sequence contained in 30Kc6. The pET-22b(+) carrying 30Kc6 was designed to express the 30K protein with a 6x His tag at its C-terminal.

### EXAMPLE 2

### Protein Expression, Purification, and Refolding

The pET-22b(+) carrying 30Kc6 without signal sequence, was introduced into E. coli strain BL21(DE3) and BL21(DE3)pLysE. The transformed bacteria were grown to OD600 of 0.5, induced with 0.5 mM isopropyl-β-D-thiogalactopyranoside(IPTG), and then incubated for 4 h. The cells were harvested by centrifugation and resuspended in 4ml of lysis buffer (10 mM Tris-HCl, 150 mM NaCl, and 1 mM EDTA, pH 8.0) containing 1 mM phenylmethylsulfonyl fluoride (PMSF) for each 100 ml of culture.

Lysozyme (0.5 mg/ml) was added and the mixture was incubated on ice for 30 min. The suspended cells were disrupted by sonication (Vibracell, 4 times, each for 15 sec) and centrifuged at 4° C. The precipitate containing inclusion bodies was solubilized in 6 M guanidine hydrochloride overnight at 4° C. This solution was loaded on a Ni²⁺-charged HisTrap column (Amersham Bioscience) and the column was washed with buffer containing 6 M urea and 20 mM imidazole several times to remove the nonspecific binding.

Refolding of the bound protein was performed in an FPLC (Bio-Rad, Biologic HR) using a linear urea reverse gradient (6 M to 0 M). The total volume and flow rate of the buffer used in the linear gradient were 30 ml and 0.5 ml/min, respectively.

Finally, the refolded protein was eluted with elution buffer containing 500 mM imidazole. The eluted 30K protein was desalted into the distilled water to remove the imidazole using a HiTrap desalting column (Amersham Bioscience) and concentrated using a lyophilizer.

### EXAMPLE 3

### Quantitation of Protein

The purity of the protein obtained was determined by scanning the 30K protein band on SDS-PAGE gel using Total Lab v1.10 (Nonlinear Dynamics). The total protein concentration was measured using a Modified Lowery Protein Determination Kit (Peterson's Modification of the Micro-Lowery Method; Sigma Chemical Co., St. Louis, Mo.).

### EXAMPLE 4

### Cell Culture for Anti-Apoptotic Activity Assay

Spodoptera frugiperda (Sf9) cells were cultivated in a Grace medium (Gibco) supplemented with 10% fetal bovine serum (FBS, Gibco), 0.35 g/L NaHCO3, and antibiotic-antimycotic (Gibco) at 28° C.

HeLa cells were cultivated in DMEM (Dulbecco's modified Eagle's medium, Gibco) supplemented with 10% fetal bovine serum (FBS, Gibco), Hepes, NaHCO3 (2.02 g/L), and penicillin/streptomycin (Gibco). The cells were incubated at 37° C. in humidified air atmosphere with 5% CO2. The recombinant 30Kc6 protein expressed in E. coli, or whole silkworm hemolymph as a control, was added to the culture medium to investigate the effects on apoptosis. Collection and pre-treatment of silkworm hemolymph has been described elsewhere in detail [E. J. Kim, W. J. Rhee, T. H. Park, Isolation and characterization of an apoptosis-inhibiting component from the hemolymph of Bombyx mori, Biochem. Biophys. Res. Commun. 285 (2001) 224-228.]. The collected hemolymph was heat-treated at 60° C. for 30 min and then chilled, and centrifuged. The supernatant filtered with a 0.2-μm membrane filter was used as a medium supplement.

Either the baculovirus AcMNPV (Autographa californica multiple nuclear polyhedrovirus) or actinomycin D (Sigma) was used as an apoptosis inducer for Sf9 cells. For the baculovirus infection, the medium was aspirated and a virus stock solution was added.

A multiplicity of infection (MOI) of 13 was used for all the experiments. After incubating for 1 h, the virus solution was replaced with the medium used before the infection. Actinomycin D dissolved in sterilized water (100 μg/ml) was used to induce apoptosis at a final concentration of 200 ng/ml in each growth medium.

Staurosporine was used as an apoptosis inducer for HeLa cells. Staurosporine dissolved in DMSO (300 μM) was used to induce apoptosis at a final concentration of 600 nM in each growth medium.

### EXAMPLE 5

N-Terminal Amino Acid Sequencing of Recombinant 30K proteins

SDS-PAGE was transferred to a PVDF (polyvinylidene difluoride) membrane in transfer buffer (192 mM glycine/25 mM Tris/20% methanol/0.037% SDS) for 90 min at 90 mA using a Bio-Rad Trans Blot SD Semidry Transfer Cell.

After the transfer, the membrane was stained with ponceau S (0.2% ponceau S in 1% acetic acid) and destained with deionized water. The stained band was then cut out and air-dried. Amino acid sequencing was carried out using the Precise Protein Sequencing System (Applied Biosystems).

### EXAMPLE 6

### Apoptosis Assay

For the assay of cell viability, cell numbers were counted under an optical microscope using a hemocytometer and viable cells were detected using the trypan blue exclusion test. Since dead cells absorb trypan blue (Sigma), they can be identified under an optical microscope.

The cell viability was defined by the ratio of the viable cell number to the total cell number. For the analysis of apoptotic cells accompanying DNA fragmentation, cell nuclei were stained with 10 μg/ml Hoechst 33258 in phosphate-buffered saline (PBS, pH7.4) for 20 min and then observed using a fluorescence microscope (TE300, Nikon) with a UV filter.

For the quantitative assay of apoptosis, flow cytometric analysis was performed. Cells were collected and washed twice with PBS (pH 7.4). The cell pellets were resuspended in cold 70% ethanol for fixation and stored at −20° C. until the FACS analysis. The fixed cells were washed with PBS, incubated with 100 g/ml RNase at 37° C. for 1 h, and stained with 50 μg/ml propidium iodide for 15 min. A FACSCalibur flow cytometer (Becton Dickinson) was used for this assay.

### EXAMPLE 7

Culture Condition of Recombinant E. coli Containing 30K Gene

The medium consisted of 20 g of yeast extract, 10 g of casamino acid, 0.24 g of MgSO4·7H20, 0.01 g of CaCl2, 3 g of KH2PO4, 2.5 g of (NH4)2HPO4, 5 g of glucose, and 200 mg of ampicillin per liter in distilled water (pH 6.8). Seed culture was grown in a 500 ml flask containing 80 ml of medium in a shaking incubator at 37° C., at 250 rpm for 12 h. Batch culture was carried out in a 2.5 L jar fermentor containing 1 L of medium.

The pH was maintained at 6.8 by adding 5N HCl and 50% (v/v) NH4OH, and the dissolved oxygen concentration was maintained above 10% air saturation by controlling the agitation speed. Isopropylthio-β-D-galactoside (IPTG) was added to the cultures to a final concentration of 1 mM, and culture continued for 20 h.

### EXAMPLE 8

### Preparation of Recombinant 30Kc12 Protein

The pET-22b(+) carrying 30Kc12 (GenBank Accession No.: X07553), instead of the pET-22b(+) carrying 30Kc6 of Example 2, is introduced into E.coli strain BL21(DE3) and BL21(DE3)pLysE. The transformed bacteria thus prepared, are treated by the process described in Example 2 to prepare the recombinant anti-apoptotic protein 30Kc12.

### EXAMPLE 9

### Preparation of Recombinant 30Kc19 Protein

The pET-22b(+) carrying 30Kc19 (GenBank Accession No.: X07554), instead of the pET-22b(+)carrying 30Kc6 of Example 2 is introduced into E.coli strain BL21(DE3) and BL21(DE3)pLysE. The transformed bacteria thus prepared, are treated by the process described in Example 2 to prepare the recombinant anti-apoptotic protein 30Kc19.

### EXAMPLE 10

### Preparation of Recombinant 30Kc21 Protein

The pET-22b(+) carrying 30Kc21 (GenBank Accession No.: X07555), instead of the pET-22b(+)carrying 30Kc6 of Example 2, is introduced into E.coli strain BL21(DE3) and BL21(DE3)pLysE. The transformed bacteria thus prepared, are treated by the process described in Example 2 to prepare the recombinant anti-apoptotic protein 30Kc21.

### EXAMPLE 11

### Preparation of Recombinant 30Kc23 Protein

The pET-22b(+) carrying 30Kc23 (GenBank Accession No.: X07556), instead of the pET-22b(+) carrying 30Kc6 of Example 2, is introduced into E.coli strain BL21(DE3) and BL21(DE3)pLysE. The transformed bacteria thus prepared, are treated by the process described in Example 2 to prepare the recombinant anti-apoptotic protein 30Kc23.

While the present invention has been described with reference to particular embodiment thereof, there can be various modifications on the basis of the spirit of the present invention.

1-19. (canceled)

20. An anti-apoptotic agent which comprises 30K protein selected from the group consisting of the proteins encoded by SEQ ID NO:1, SEQ ID NO:2, SEQ ID NO:3, SEQ ID NO:4, and SEQ ID NO:5.

21. The anti-apoptotic agent of claim 20, wherein the 30K protein comprises 30Kc6 protein encoded by the DNA of SEQ ID NO:1.

22. The anti-apoptotic agent of claim 20, wherein the 30 K protein comprises 30Kc12 protein encoded by the DNA of SEQ ID NO:2.

23. The anti-apoptotic agent of claim 20, wherein the 30 K protein comprises 30Kc19 protein encoded by the DNA of SEQ ID NO:3.

24. The anti-apoptotic agent of claim 20, wherein the 30 K protein comprises 30Kc21 protein encoded by the DNA of SEQ ID NO:4.

25. The anti-apoptotic agent of claim 20, wherein the 30 K protein comprises 30Kc23 protein encoded by the DNA of SEQ ID NO:5.

26. A method of inhibiting apoptosis of a cell by transfecting the cell using a recombinant expression vector comprising a nucleic acid selected from the group consisting of SEQ ID NO:1, SEQ ID NO:2, SEQ ID NO:3, SEQ ID NO:4, and SEQ ID NO:5.

27. The method of inhibiting apoptosis of a cell of claim 26, wherein the recombinant expression vector is a nucleic acid of SEQ ID NO:1.

28. The method of inhibiting apoptosis of a cell of claim 26, wherein the recombinant expression vector is a nucleic acid of SEQ ID NO:2.

29. The method of inhibiting apoptosis of a cell of claim 26, wherein the recombinant expression vector is a nucleic acid of SEQ ID NO:3.

30. The method of inhibiting apoptosis of a cell of claim 26, wherein the recombinant expression vector is a nucleic acid of SEQ ID NO:4.

31. The method of inhibiting apoptosis of a cell of claim 26, wherein the recombinant expression vector is a nucleic acid of SEQ ID NO:5.

32. A method for inhibiting apoptosis of a cell by adding to a cell culture medium a protein selected from a 30Kc6 protein, a 30Kc12 protein, a 30Kc19 protein, a 30Kc21 protein and a 30Kc23 protein.

33. The method according to claim 32, wherein 30Kc6 protein is added.

34. The method according to claim 32, wherein 30Kc12 protein is added.

35. The method according to claim 32, wherein 30Kc19 protein is added.

36. The method according to claim 32, wherein 30Kc21 protein is added.

37. The method according to claim 32, wherein 30Kc23 protein is added.


SEQUENCE LISTING (10 sequences)

SEQ ID NO: 1 - DNA, 771 bp, Bombyx mori
atgagactgactttgtttgccttcgtcctcgccgtgtgtgcgctggcttctaacgccacacttgcaccaagaactgatgacgtactggcagagcagctgtatatgagtgtcgtcattggtgaatacgagaccgctatcgccaaatgctctgaatatctgaaggaaaagaagggagaggttatcaaggaagccgtgaagcgtctgatcgaaaacggcaagaggaacaccatggacttcgcctaccagttatggacaaaggatggaaaggaaatcgtcaaatcttacttccccatccagtttagagtgatcttcaccgagcagactgtcaagctcataaacaaaagggaccatcacgccctcaagttgatcgaccaacaaaaccacaacaaaattgcattcggtgactccaaagacaaaaccagcaagaaagtctcctggaagtttacccccgtgttggaaaacaacagagtatacttcaagatcatgtccaccgaagacaaacagtacctgaagctcgataacacgaaaggttctagtgatgaccgtatcatctacggtgatagcaccgctgacaccttcaaacaccactggtaccttgagccctccatgtacgaaagcgacgtcatgttcttcgtctacaaccgagagtacaacagtgttatgacacttgatgaagatatggccgccaacgaagaccgtgaagccttggggcacagcggagaagtttccggttatccccaactttttgcatggtacatcgtcccctactaa

SEQ ID NO: 2 - DNA, 795 bp, Bombyx mori
atgaaacttctcgttgtgttcgcaatgtgcgtgcctgccgccagcgccggcgtcgtggaactatccgcggacagcatgagcccttctaaccaagacctcgaggacaaactgtacaacagcatcctcaccggtgactacgacagtgctgtacgtaagagcttggaatacgagagccaaggccagggcagcatcgttcaaaatgtagttaacaatctgattattgacaagagacggaacaccatggagtactgctacaagctgtgggtcggcaacggacaggatattgtcaaaaagtacttcccattaagctttagactcatcatggccggaaactacgtcaagctcatttacagaaactacaacctcgctctgaagctcggttccacaaccaatccctcgaatgagagaattgcctacggcgatggtgttgacaagcacactgacctcgtcagttggaagttcattaccttgtgggagaacaacagagtgtacttcaaggcccacaacactaagtacaaccagtacttgaagatgagtacgtcgacttgcaactgcaacgctcgggaccgtgttgtatacggcggcaacagcgctgacagcaccagggagcaatggttcttccagcccgccaagtacgaaaacgacgtcctgttcttcatctacaatcgccaattcaacgatgccttggagctcggtacgatcgtgaacgcctcgggagaccgcaaggccgttggacacgatggtgaagtcgccggtcttcctgacatctactcgtggttcattacacctttctaa

SEQ ID NO: 3 - DNA, 765 bp, Bombyx mori
atgaagcccgctatagttattctatgtcttttcgtggcatctctgtatgctgcagattccgacgtccctaacgacattctggaggagcagctttacaatagcgtcgtcgtcgccgattacgacagtgcggttgaaaagagcaagcatttatacgaggagaagaagagcgaagtcatcacaaatgtcgtgaacaaactgatacgaaacaacaagatgaactgcatggagtacgcctatcaactttggctccagggctccaaggacatcgtccgggattgtttcccagttgagttcagacttatcttcgccgaaaacgcgattaagcttatgtacaagcgcgacggtctcgctttgacgctgagcaatgatgttcaaggcgacgatggcagacctgcctacggcaaggacaagacaagcccgagagtcagctggaagttaatcgctctgtgggagaacaacaaggtctacttcaagatcttgaacactgaacgtaaccaatacttggtattgggagtcggcactaactggaacggcgaccatatggccttcggagtcaacagcgtcgatagtttcagagcccagtggtacctgcagcctgctaagtacgacaatgacgtcttgttctacatctacaaccgtgaatacagcaaggctttgacactgtcgaggacggttgagccctcgggtcaccgcatggcctggggatacaacggcagagtaatcggaagtcccgaacattacgcttggggtataaaggctttctaa

SEQ ID NO: 4 - DNA, 756 bp, Bombyx mori
atgaaatttgtcgtggtgttcgcgtcgtgcgtgctcgccgtcagcgccggcgtcactgaaatgtccgcggcaagcatgagcagttctaacaaagaactcgaggagaaactgtacaacagcatcctcaccggtgactacgacagcgctgtccgtcagagcttggaatacgagaaccaaggcaagggctccatcatccagaatgtagttaacaacctgatcattgacggaagtcggaacaccatggagtactgctacaagctgtgggtcggcaacggacagcacattgtcagaaagtacttcccctataactttagactcatcatggccggaaacttcgttaagctcatttacagaaactacaacctcgctctgaagctcggccccactcttgatcccgcgaacgagagacttgcatacggcgatggtaaggaaaagaacagcgacctcatcagttggaagtctcattaccttgtgggagaacaacatagtgtacttcaagatccaccaacactaagctacaaccagtacttgaaactaagttcgactactgactgcaacactcaagaccgtattatattcggcaccaacaccgccgacaccaccagggagcagtggttcctccagcccacgaagtacgaaaacgacgtcctgttcttcatctacaatcgcgaggtacaacgagtggctttgaagctcggtaggattgtggacgcttcgggagaccgtagtggcatttggacacgatggatgaagtag

SEQ ID NO: 5 - DNA, 795 bp, Bombyx mori
atgaaatttctcgtggtgttcgcggtcgtgcgtgcctgcgtcacgccggcgtgcgctgaaatgtccgcggtaagcatgagcagttctaacaaagaactcgaggagaaactgtacaacagcatcctcaccggcgactacgacagtgctgtccgccagagcttggaatacgagagccaaggcaagggctccatcatccagaatgtagttaacaacctgatcattgacaagagacggaacaccatggagtactgctacaagctgtgggtcggcaacggacaggaaattgttagaaagtacttcccattaaactttagactcatcatggccggaaactatgtcaagatcatttacagaaactacaacctcgctctgaagctcggttccacaaccaatccctcgaatgagagaattgcctacggcgatggtgtagacaagcatactgaactcgtcagttggaagttcattaccttgtgggagaacaacagagtgtacttcaagatccacaacactaagtacaaccagtacttgaagatgagtacgacgacttgcaactgcaacagtcgcgaccgtgttgtatacggcggcaacagcgctgacagcaccagggagcaatggttcttccagcccgccaagtacgaaaacgacgtcctgttcttcatctacaatcgccaattcaacgatgccttggagctcggtacgatcgtgaacgcctcgggagaccgcaaggccgttggacacgatggtgaagtcgccggtcttcctgacatctactcgtggttcattacacctttctaa

SEQ ID NO: 6 - protein, 238 aa, Bombyx mori
MTLAPRTDDVLAEQLYMSVVIGEYETAIAKCSEYLKEKKGEVIKEAVKRLIENGKRNTMDFAYQLWTKDGKEIVKSYFPIQFRVIFTEQTVKLINKRDHHALKLIDQQNHNKIAFGDSKDKTSKKVSWKFTPVLENNRVYFKIMSTEDKQYLKLDNTKGSSDDRIIYGDSTADTFKHHWYLEPSMYESDVMFFVYNREYNSVMTLDEDMAANEDREALGHSGEVSGYPQLFAWYIVPY

SEQ ID NO: 7 - protein, 249 aa, Bombyx mori
MGVVELSADSMSPSNQDLEDKLYNSILTGDYDSAVRKSLEYESQGQGSIVQNVVNNLIIDKRRNTMEYCYKLWVGNGQDIVKKYFPLSFRLIMAGNYVKLIYRNYNLALKLGSTTNPSNERIAYGDGVDKHTDLVSWKFITLWENNRVYFKAHNTKYNQYLKMSTSTCNCNARDRVVYGGNSADSTREQWFFQPAKYENDVLFFIYNRQFNDALELGTIVNASGDRKAVGHDGEVAGLPDIYSWFITPF

SEQ ID NO: 8 - protein, 238 aa, Bombyx mori
MADSDVPNDILEEQLYNSVVVADYDSAVEKSKHLYEEKKSEVITNVVNKLIRNNKMNCMEYAYQLWLQGSKDIVRDCFPVEFRLIFAENAIKLMYKRDGLALTLSNDVQGDDGRPAYGKDKTSPRVSWKLIALWENNKVYFKILNTERNQYLVLGVGTNWNGDHMAFGVNSVDSFRAQWYLQPAKYDNDVLFYIYNREYSKALTLSRTVEPSGHRMAWGYNGRVIGSPEHYAWGIKAF

SEQ ID NO: 9 - protein, 236 aa, Bombyx mori
MGVTEMSAASMSSSNKELEEKLYNSILTGDYDSAVRQSLEYENQGKGSIIQNVVNNLIIDGSRNTMEYCYKLWVGNGQHIVRKYFPYNFRLIMAGNFVKLIYRNYNLALKLGPTLDPANERLAYGDGKEKNSDLISWKSHYLVGEQHSVLQDPPTLSYNQYLKLSSTTDCNTQDRIIFGTNTADTTREQWFLQPTKYENDVLFFIYNREVQRVALKLGRIVDASGDRSGIWTRWMK

SEQ ID NO: 10 - protein, 242 aa, Bombyx mori
MVSMSSSNKELEEKLYNSILTGDYDSAVRQSLEYESQGKGSIIQNVVNNLIIDKRRNTMEYCYKLWVGNGQEIVRKYFPLNFRLIMAGNYVKIIYRNYNLALKLGSTTNPSNERIAYGDGVDKHTELVSWKFITLWENNRVYFKIHNTKYNQYLKMSTTTCNCNSRDRVVYGGNSADSTREQWFFQPAKYENDVLFFIYNRQFNDALELGTIVNASGDRKAVGHDGEVAGLPDIYSWFITPF